(19) United States
(12) Patent Application Publication
(10) Pub. No.: US 2015/0088092 A1
(43) Pub. Date: Mar. 26, 2015

(54) Drug Delivery Device and Method for Detection of End-Of-Dose Condition

(71) Applicant: Novo Nordisk A/S, Bagsvaerd (DK)

(72) Inventors: Per Einar Pontus Holm, Tygelsjoe (SE); Jens A. Munk, Oelstykke (DK)

(73) Assignee: NOVO NORDISK A/S, Bagsvaerd (DK)

(21) Appl. No.: 14/395,976

(22) PCT Filed: Apr. 24, 2013
(86) PCT No.: PCT/EP2013/058456

§ 371 Date: Oct. 21, 2014

Related U.S. Application Data
(60) Provisional application No. 61/639,393, filed on Apr. 27, 2012.

(30) Foreign Application Priority Data
Apr. 24, 2012 (EP) 12165249.9

(51) Int. Cl. A61M 5/50

(52) U.S. Cl. CPC A61M 5/5086; A61M 2205/18; A61M 2205/3306
USPC 604/506; 604/111

(57) ABSTRACT

Method for detecting an end-of-dose condition in a pressurized system, comprising the steps of (a) providing a system having a fluid-filled variable volume reservoir comprising an elastically deformable portion and an outlet, pressurizing means, and means for detecting a property of the elastically deformable portion which varies with the internal pressure in the reservoir, (b) pressurizing the reservoir to expel a desired amount of fluid by applying a force, thereby deforming the elastically deformable portion, (c) measuring, after the actuating step, a property related to the elastically deformable portion as it regains its initial configuration, (d) comparing a value for the measured property with one or more threshold values, and (e) providing a user with an indication when a given threshold value has been reached, and/or when a given threshold value has not been reached within a given period of time.

The present invention generally relates to a drug delivery device adapted to receive a drug filled cartridge and expel a dose therefrom. More specifically, the invention relates to the issue of informing a user of user-relevant events after detection of an end-of-dose condition.

### BACKGROUND OF THE INVENTION

In the disclosure of the present invention reference is mostly made to the treatment of diabetes, however, this is only an exemplary use of the present invention.

The most common type of durable drug delivery devices adapted to receive a drug filled cartridge and expel a set dose therefrom are driven by manual means or by a spring energized during dose setting, the cartridge being of the type comprising an axially displaceable piston having an initial proximal position. The device may be pen-formed or in the form of a more box-shaped so-called doser. In order to improve convenience, user-friendliness and provide additional features, e.g. detection and storing of expelling data, drug delivery devices have been provided with electrically driven means, typically in the form of an electronically controlled motor driving a piston rod through a gear arrangement, e.g. as shown in U.S. Pat. No. 6,514,230 and US 2011/306927. The same arrangement is also used in infusion pumps, e.g. as shown in U.S. Pat. No. 7,193,521. When a new cartridge is loaded into a motor-driven drug delivery device the piston is first moved proximally allowing a full cartridge to be inserted after which the piston rod is moved into contact with the cartridge piston to bring the drug delivery device in an operational state. The loading of a new cartridge may be performed manually, i.e. the user actuates buttons moving the piston rod back and forth, or it may be partly or fully automated, e.g. the device detects that a cartridge cover is opened and moves back the piston rod, this allowing the user to remove the used cartridge, insert a new and close the cover. When the device detects that the cover has been closed and a cartridge is inserted the piston rod is automatically advanced into engagement with the piston where after the device is ready for use or further initial operations, e.g. a user operated air shot to drive out air in the cartridge or an attached needle.

As indicated above, after a cartridge has been loaded proper operation of the system can be checked by performing an air shot until visible drug appears at the tip of the needle. Such a check can be performed both initially when a new cartridge has been loaded as well as each time a new needle is mounted to check that the needle is not defect or blocked, this also being the recommend when mounting a new needle. Although it is not recommend using a needle more than once, proper operation of a used needle should also be checked prior to each drug delivery. However, just as all users do not follow these recommendations, a needle may also be blocked during use, e.g. either by external matter during skin insertion or internal matter contained in the cartridge, e.g. crystals formed in the drug or dried-up drug from previous injections.

Correspondingly, for a motorized drug delivery device detection systems have been proposed adapted to detect a blocked needle condition or, for a similar type of device, a blocked infusion needle or infusion catheter in a drug delivery pump for continuous drug administration. Such systems have been based on the fact that when the infusion conduit, e.g. needle or catheter, downstream of the cartridge is blocked, then a blockage can be detected directly or indirectly by detecting pressure-build up in the system. For example, when resistance to piston movement increases the electric current to the motor in many control systems will increase which can then be detected and used to determine pressure rise and thus assumed needle or catheter blockage. Alternatively, US 2003/0073954 discloses the use of electronic switches and strain gauge sensors to detect pressure build up in an occluded system. US 2011/0172594 discloses that an occlusion detection sensor can be configured to detect alteration of a shape of the occlusion detection portion, e.g. by means a pressure sensor, a capacitance sensor, an optical sensor, or other type of sensor. However, in some systems and under some conditions, an occlusion may not result in a change of parameters which are sufficient to detect an actual occlusion. For example, in cartridge based system in which a set dose is small, e.g. less than 5 IU for a traditional 3 ml 100 Um/ml insulin cartridge, then the corresponding axial displacement of the piston drive member, e.g. the piston rod, will in most cases be absorbed by the cartridge piston which typically is manufactured from a polymeric rubber material. In this way the piston driver may be fully forwarded to an end-of-dose position merely deforming the piston and thus without a corresponding pressure or tension build-up in the drive system. Indeed, if a further elastic system such as a catheter infusion line is arranged between the cartridge and infusion needle then further piston driver movement may be absorbed without a corresponding pressure or tension build-up in the drive system.

Further, injection systems using an elastic rubber piston is characterised in that the piston will be more or less compressed when the piston drive is advanced. When the piston drive stops moving when it has reached its end-of-dose position, the piston will expand to its original form. The selected dose is not fully delivered until the piston has reached its form just before starting the injection. How much the piston is compressed depends mainly on the friction between the piston and the glass cartridge plus the counter pressure that builds up when the liquid has to pass through the very thin needle. This is the main reason why the user is recommended to keep the needle inserted for some period—typically six seconds. Removing the needle before six seconds has elapsed might lead to an under dose of up to 1 IU which could be substantial for small doses. Waiting six seconds is cumbersome and some users might ignore it with the risk of an under dose as a consequence. In most cases more than 95% of the dosage is delivered within 1-2 seconds after stopping the piston drive.

Having regard to the above, it is an object of the present invention to provide a method and a motorized drug delivery device adapted to receive a drug-filled cartridge, and which are adapted to detect one or more user-relevant end-of-dose conditions (events), i.e. conditions following the formal end of out-dosing. The conditions should be detected in a simple and effective way, the arrangement being sensitive, cost-effective and reliable. A specific object of the invention is to provide a system which will detect that a given dose has been (almost) fully delivered which in most cases will be before six seconds has elapsed. A further specific object of the invention is to provide a system which will detect an outlet blockage, e.g. a blocked needle.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in accordance with a first aspect of the invention a method of detecting an end-of-dose condition in a pressurized system is provided, the method comprising the step of providing a system comprising a fluid-filled variable volume reservoir comprising an elastically deformable portion and an outlet, the elastically deformable portion having an initial configuration, means for pressurizing the interior of the reservoir to thereby expel fluid through the outlet, and means for measuring a property of the elastically deformable portion which varies with the internal pressure in the reservoir. The method comprises the further steps of actuating the pressurizing means to pressurize the reservoir to expel a desired amount of fluid by applying a force, thereby deforming the elastically deformable portion, measuring, after the actuating step, a property related to the elastically deformable portion as it regains its initial configuration, comparing a value for the measured property with one or more threshold values, and providing a user with an indication when a given threshold value has been reached, and/or when a given threshold value has not been reached within a given period of time.

In this way a method is provided adapted to detect user-relevant “end-of-dose” conditions taking place after actuation of the pressurizing means has stopped, e.g. an indication is provided that expelling of the desired amount of fluid has (almost) ended when the given threshold value has been reached, and/or an indication is provided that an occlusion state applies to the reservoir outlet when a given threshold value has not been reached within a given period of time.

In the context of the present invention the threshold value to be reached may not only be a specific value but may also be in the form of a rate of change of the measured property.

Values for the measured property after the actuating step may be determined based on a number of discrete measurements or continuous measurement of the property. Indeed, also continuous measurement normally takes place at a given sampling rate.

The one or more threshold values may be determined based on a reference value such as a value determined prior to pressurizing the reservoir, or a value determined at the end of the action pressurizing the reservoir. For example, a reference value may have been determined just prior to pressurizing the reservoir. Based on this value a difference to the end-of-dose value can be calculated which again can be used to calculate a threshold value. For example, the threshold value may correspond to when 90% of the difference between the two values has been regained, this indicating when the set dose has been almost fully expelled, typically after 1-2 seconds. Correspondingly, when a given threshold value, e.g. the same as above, has not been reached within a given period, e.g. 6 seconds, an occlusion state may be detected. Other values may be used.

The provided system may further comprise a reflecting surface adapted to reflect light, a light source directing light against the reflecting surface, and a light sensor adapted to measure light from the light source reflected from the reflecting surface, a measured light value varying with the deformation of the elastically deformable portion, wherein the measured property is light reflected from the reflecting surface.

The reservoir of the provided system may comprise a main portion having a general cylindrical configuration, and an axially displaceable piston arranged in the main portion, the piston comprising at least a part of the elastically deformable portion, and the reflecting surface. Alternatively to reflected light, the measured property may be pressure in the reservoir or tension in a part of the pressurizing means.

In accordance with a second aspect of the invention a drug delivery device is provided comprising a drug-filled cartridge in a loaded position or a compartment adapted to receive a drug-filled cartridge in a loaded position, the cartridge comprising a generally cylindrical body portion, an axially displaceable elastically deformable piston with a proximal portion comprising a surface adapted to reflect light, and a distal outlet portion adapted to be arranged in fluid communication with a flow conduit. The device further comprises an expelling assembly comprising an axially displaceable piston drive member adapted to engage the proximal piston portion of a loaded cartridge, the piston drive member being moveable in a distal direction to thereby expel drug from a loaded cartridge, the piston thereby being elastically deformed by the piston drive member from its initial configuration, a light source providing a light directed at least in part against the proximal piston portion of a loaded cartridge, a light sensor adapted to measure light from the light source reflected from the proximal piston portion, the light sensor being arranged to provide a measured value varying with the deformation of the piston, and a controller coupled to the light sensor. The controller is configured to detect (with a cartridge in a loaded position) an end-of-stroke state when the piston drive member has just been moved to a position corresponding to expelling of a set dose, the end-of-stroke state being characterized by the piston being elastically deformed by the piston drive member corresponding to a build-up of pressure in the cartridge, measure, after detection of the end-of-stroke state, light from the light source reflected from the proximal piston portion as the piston regains its initial configuration, and compare a measured light value with one or more threshold values. The controller is further configured to provide a user with an indication (e.g. that expelling of the desired amount of fluid has ended) when a given threshold value has been reached, and/or when a given threshold value has not been reached within a given period of time, e.g. an indication that an occlusion state applies to the reservoir outlet.

The threshold may be determined based on a reference value being one of a value determined prior to pressurizing the reservoir, and a value determined at the end of the action pressurizing the reservoir. The light source and the light sensor provide at least in part a proximity sensor system adapted to provide an output indicative of the distance between the proximal piston portion and the piston drive member.

In exemplary embodiments the expelling assembly is electronically controlled, the controller being coupled to the expelling assembly and adapted to control the expelling assembly in order to expel a set dose. The light source and/or the light sensor may be coupled to and move with the piston drive member.

In accordance with a further aspect of the invention an exemplary drug delivery device is provided comprising a drug-filled cartridge in a loaded position or a compartment adapted to receive a drug-filled cartridge in a loaded position, the cartridge comprising a body portion, an axially displaceable elastically deformable piston with a proximal portion comprising a surface adapted to reflect light, and a distal outlet portion adapted to be arranged in fluid communication with a flow conduit, an expelling assembly comprising an axially displaceable piston drive member adapted to engage the proximal piston portion of a loaded cartridge, the piston drive member being moveable in a distal direction to thereby expel drug from a loaded cartridge, the piston thereby being elastically deformed by the piston drive member. A light source provides a light directed at least in part against the proximal piston portion of a loaded cartridge, a light sensor is adapted to measure light from the light source reflected from the proximal piston portion, the light sensor being arranged to provide a measured value varying with the deformation of the piston. A controller is coupled to the light sensor. With a cartridge in a loaded position the controller is configured to: Detect a first “end-of-stroke” state when the piston drive member has just been moved to a position corresponding to expelling of a set dose, the first state being characterized by the piston being elastically deformed by the piston drive member corresponding to a build-up of pressure in the cartridge. Then after a given amount of time determine an end-of-dose sensor value based on light reflected from the proximal piston portion, and compare the end-of-dose sensor value with a reference value. Based on the difference between the end-of-dose sensor value and the reference value, the controller is configured to determine (i) a non-occlusion state when the elastically deformed piston has relaxed to such a degree that it is indicative of a reduction in the pressure build-up in the cartridge due to drug being expelled from the cartridge via a connected flow conduit by energy stored in the deformed piston, or (ii) an occlusion state when the elastically deformed piston has failed to relax thereby being indicative of a connected flow conduit being occluded. By this arrangement it is possible to detect an occlusion state for the delivery of relatively small dose amounts of drug from a drug delivery device. Alternatively a sound generator and an acoustic sensor could be used instead of the light source and light sensor.

The expelling assembly may be mechanical or electronically controlled, the controller being coupled to the expelling assembly and adapted to control the expelling assembly in order to expel a set dose. The light source and/or the light sensor may be coupled to and move with the piston drive member.

The reference sensor value may be determined in different ways, e.g. it may be determined prior to the piston drive being moved to expel a set dose or when the piston drive has just been moved to the position corresponding to expelling of a set dose. The reference sensor value may also be a pre-set value or it may be calculated from one or more determined values. Alternatively, instead of determining an end-of-dose sensor value the sensor output may be analysed over a period of time and used to determine an occlusion or non-occlusion state, e.g. by determining the rate of sensor value change.

The light source and the light sensor may also provide at least in part a proximity sensor system adapted to provide an output indicative of the distance between the proximal piston portion and the piston drive member. Such an output may be used to control movement of the piston drive member during initial cartridge loading.

The controller may be in the form of a CPU or a microcontroller as well as their supporting components or any other configuration of electronic components suitable for the described functionality.

The drug delivery device as described above may be provided in combination with a drug-filled cartridge comprising an axially displaceable piston having a proximal portion, the proximal portion comprising a surface adapted to reflect light as well as an engagement portion adapted to engage the piston drive member.

In a yet further aspect of the invention a method of detecting an obstruction of an outlet from a pressurized system is provided, comprising the steps of (a) providing a system comprising a fluid-filled variable volume reservoir comprising an elastically deformable portion and an outlet, means for pressurizing the interior of the reservoir to thereby expel fluid through the outlet, and means for detecting a property of the elastically deformable portion which varies with the internal pressure in the reservoir, (b) actuating the pressurizing means to pressurize the reservoir to expel a desired amount of fluid by applying a force, thereby deforming the elastically deformable portion, (c) after or over a given time after the actuating step measuring a property related to the elastically deformable portion, (d) comparing the measured property with a reference value, and (e) based on the result of the comparing step decide whether (i) a non-occlusion state or (ii) an occlusion state applies to the reservoir outlet.

More than one reference value may be used to improve accuracy of the system. By the described method it may be possible to detect an occlusion based on a pressure build-up which is normally associated with normal operation of a system.

A given reference value may be one of a rate of change of the measured property, a sensor value determined prior to pressurizing the reservoir, a sensor value determined at the end of the action pressurizing the reservoir, and a pre-set value.

In exemplary embodiments the provided system further comprises a reflecting surface adapted to reflect light, a light source directing light against the reflecting surface, and a light sensor adapted to measure light from the light source reflected from the reflecting surface, a measured value varying with the deformation of the elastically deformable portion, wherein the measured property is light reflected from the reflecting surface. Alternatively a sound generator and an acoustic sensor could be used instead of the light source and light sensor.

The reservoir of the provided system may further comprise a main portion having a general cylindrical configuration, and an axially displaceable piston arranged in the main portion, the piston comprising at least a part of the elastically deformable portion, and the reflecting surface.

Alternatively, other properties could be measured. For example, if an elastically deformable piston is made at least in part by a material having a dielectric constant different from air then a sensor system could be adapted to measure changes in deformation using capacitance. If an elastically deformable piston is made at least in part by a material having magnetic properties which changes with deformation a sensor system could be adapted to measure changes in deformation using such properties. As a further alternative an elastically deformable piston may be provided with a strain gauge allowing deformation to be measured.

In a further aspect of the invention a drug delivery device is provided comprising a housing, means for receiving and holding a drug-filled cartridge in a loaded position, the cartridge having a cylindrical configuration comprising an axially displaceable piston, the cartridge being adapted to be fitted with a hollow needle in fluid communication with the drug, a needle fitted on a loaded cartridge projecting from a distal end of the device, motor driven drug expelling means comprising a piston drive adapted to engage the piston of a loaded cartridge, the piston drive being axially moveable in a distal direction and along a general axis to thereby expel drug from a loaded cartridge, position detecting means adapted to measure the inclination of the general axis relative to a vertical position, a flow check state being determined when the position detecting means measures that the general axis is oriented within a pre-defined range relative to the vertical position, and a controller coupled to the position detecting means and adapted to control the expelling means. In such a device the controller is adapted to move the piston drive in the distal direction when a flow check state is determined, whereby, when the piston drive is engagement with the piston of a loaded cartridge fitted with a hollow needle, drug is expelled from the cartridge. In this way a user can perform an air-shot in an easy and convenient way without having to operate any buttons.

The drug delivery device may further comprise a cap releasably mountable on the drug delivery device and adapted to cover a fitted needle, as well as cap sensing means coupled to the controller and adapted to detect either a cap-off state or a cap-on state, wherein, when a cap-on state is detected, the controller will not move the piston drive based on input from the position detecting means. The controller may, when the conditions are met, start moving the piston drive after a pre-determined time delay. To avoid a jet of expelled drug, the controller may forward the piston drive in smaller steps, this allowing a droplet to neatly form at the distal tip of an attached needle. Proximity sensing means may be coupled to the controller and adapted to measure the distance of the distal end of the device from a skin surface, a proximity state being determined when a distance within a pre-defined range is measured, such that, when a flow check state and a proximity state are detected, the controller will not move the piston drive, this preventing misuse of the automated expelling. Further restraints to automated expelling may be implemented, e.g. maximum size and number of expelled air-shot doses.

In a yet further aspect of the invention a drug delivery device is provided comprising a housing, means for receiving and holding a drug-filled cartridge in a loaded position, the cartridge being adapted to be fitted with a hollow needle in fluid communication with the drug, a needle fitted on a loaded cartridge projecting or being adapted to project from a distal end of the device, and expelling means adapted to engage a loaded cartridge. The device further comprises detecting means adapted to measure and determine a delivery condition, and a controller coupled to the detecting means and adapted to control the expelling means, wherein the controller is adapted to actuate the expelling means when a delivery condition is determined, whereby, when a hollow needle is connected to the cartridge, drug is expelled from the cartridge. By this arrangement use of the drug delivery is simplified as the user no longer has to release or actuate the expelling mechanism.

The needle may be mounted in a fixed position projecting from the housing and thus adapted to be inserted manually, or it may be arranged in an initially retracted position and then inserted automatically prior to expelling of drug, e.g. initiated by detection of a delivery condition. The detecting means may be adapted to measure e.g. proximity of the device to a surface (e.g. light reflection as described above or sound reflection), actuation of a mechanical member (e.g. a mechanical switch), skin contact (e.g. based on properties unique to a skin surface), and needle insertion when performed manually (e.g. based on the needle having or providing a property that changes when it is inserted). The cartridge may comprise an axially displaceable piston and the expelling means may comprise a corresponding piston drive member.

As used herein, the term “drug” is meant to encompass any flowable medicine formulation capable of being passed through a delivery means such as a cannula or hollow needle in a controlled manner, such as a liquid, solution, gel or fine suspension, and containing one or more drug agents. Representative drugs include pharmaceuticals such as peptides (e.g. insulins, insulin containing drugs, GLP-1 containing drugs as well as derivates thereof), proteins, and hormones, biologically derived or active agents, hormonal and gene based agents, nutritional formulas and other substances in both solid (dispensed) or liquid form. In the description of exemplary embodiments reference will be made to the use of insulin containing drugs, this including analogues thereof as well as combinations with one or more other drugs.

### BRIEF DESCRIPTION OF THE DRAWINGS

In the following exemplary embodiments of the invention will be further described with reference to the drawings, wherein

FIG. 1 shows a schematic representation of components of a drug delivery device in a loading state,

FIG. 2 shows the drug delivery device in a loaded state,

FIG. 3 shows the drug delivery device in a pressurized state, and

FIGS. 4 and 5 show data from experiments made with set-ups similar to the system shown in FIG. 3.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not to an actual situation of use. The shown figures are schematic representations for which reason the configuration of the different structures as well as their relative dimensions are intended to serve illustrative purposes only.

An exemplary embodiment of the invention is implemented in an electronically controlled motorized drug delivery device adapted to receive a drug-filled cartridge comprising an outlet and an axially displaceable piston, the device comprising a housing, a compartment adapted to receive and hold a cartridge, an electronically controlled expelling assembly comprising an axially displaceable piston drive member adapted to engage the piston of a loaded cartridge, a controller coupled to the expelling assembly and configured to control the expelling assembly to move the piston in a distal direction to thereby expel drug from a loaded cartridge, an electrical energy source for the controller and the expelling assembly, as well as user-operated input means for setting a desired dose of drug to be expelled. Such a drug delivery device is known per se, e.g. in the form of a compact doser incorporating a flexible piston rod as disclosed in U.S. Pat. No. 6,514,230, a pen-formed device incorporating a rigid piston rod as disclosed in US 2011/306927 or an infusion pump as disclosed in U.S. Pat. No. 7,193,521 which are all hereby incorporated by reference.

As also described initially, a motorized drug delivery device is adapted to have an empty cartridge replaced with a new cartridge in which course the piston rod is normally moved proximally to a loading position by the motorized expelling assembly, which action may be activated by e.g. a cover being opened or a button being pressed. Correspondingly, when a new cartridge has been inserted the piston rod is normally moved automatically distally to a loaded position in contact with the piston by the motorized expelling assembly, which action may be activated by a cover being closed or a button being pressed. The device may further be provided with means for detecting that a cartridge has been loaded to prevent an initialization procedure when no cartridge is mounted.

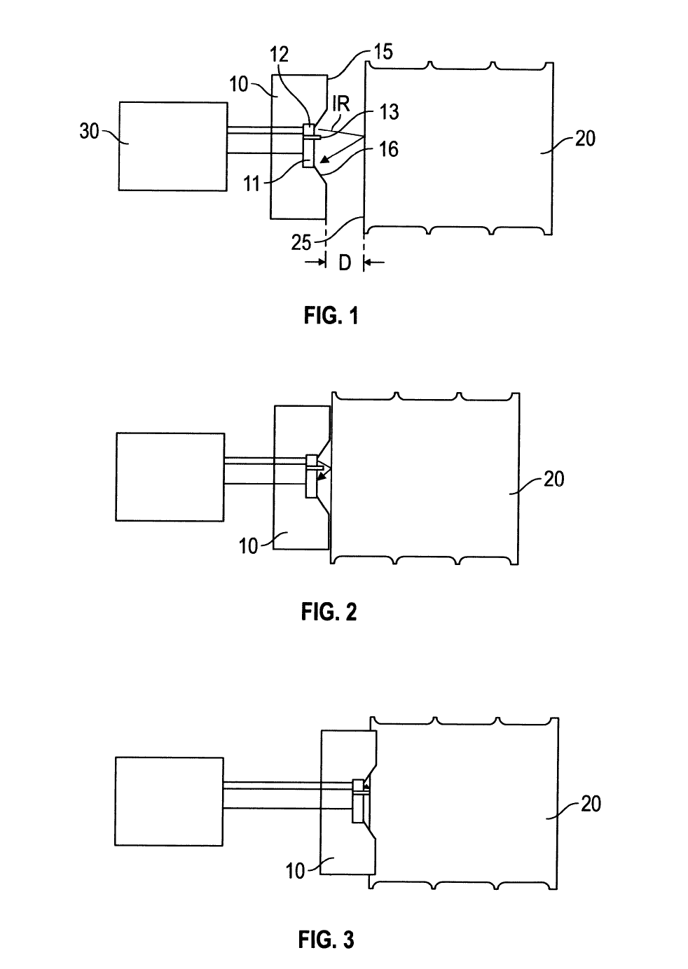

Referring to FIG. 1 a schematic representation of components of a drug delivery device is shown, the figure only showing those structures relevant for illustrating an embodiment of the invention. More specifically, the drug delivery device comprises a piston drive member in the form of a piston rod washer 10 mounted on a piston rod (now shown) and driven by an electronically controlled motorized mechanism (not shown), a piston 20 mounted in a cartridge (not shown), and a controller 30. The piston rod washer comprises a distal surface 15 adapted to engage a proximal surface 25 on the piston, the distal surface being provided with a central cavity 16 in which a light sensor 11 and a light source 12 in the form of a IR LED are arranged next to each other with a barrier member 13 being mounted there between. The IR LED is arranged to direct IR light towards the proximal surface of the piston and the light sensor is arranged to detect the reflected light therefrom, the barrier preventing or limiting direct light from the IR LED to reach the sensor.

In FIG. 1 the piston rod washer has been advanced to a distance corresponding to approximately 2 mm from the piston, this allowing both reflected IR light and backlight (as well as ambient light) to reach the sensor. This state does not correspond to an operational state in accordance with aspects of the present invention, however, the reflected and sensed light may be used to control piston rod advancement during a loading procedure, however, this aspect is not part of the present invention but may represent an optional use of the provided structures.

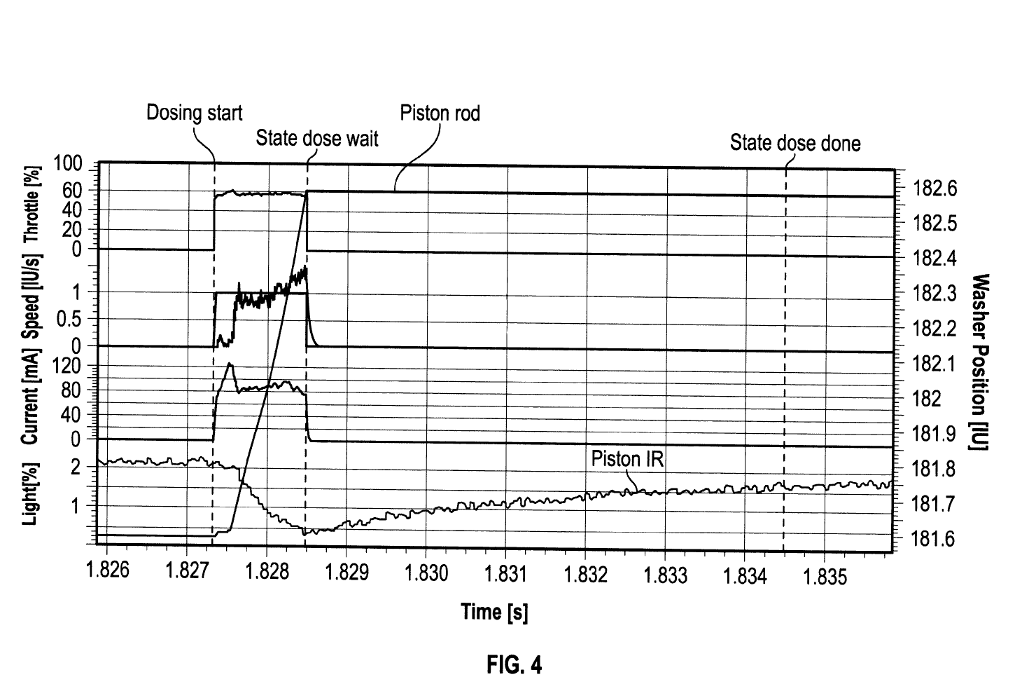

In FIG. 2 the piston rod washer has been further advanced and has now engaged the piston without deforming the latter, this state corresponding to the condition after a set dose of drug has been fully expelled from the cartridge. In this state reflected IR light from the piston is almost prevented from reaching the sensor, however, this “leak” of reflected light is intended as the measured level of light may be used as a reference for detecting an occlusion state as will be described below.

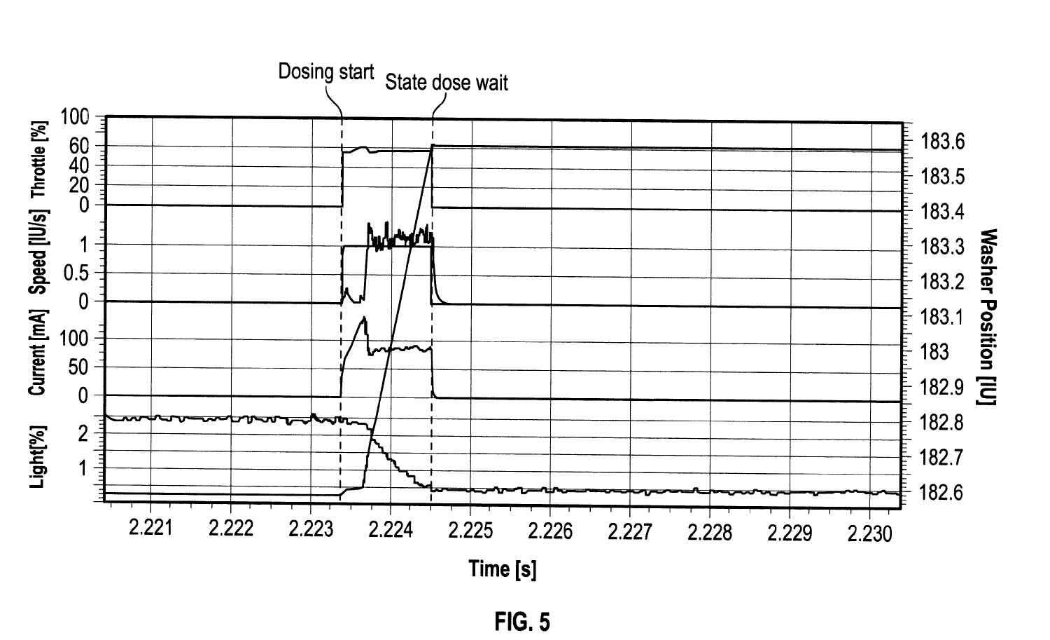

FIG. 3 shows the state just after the piston rod washer has just been moved to an end-of-stroke position corresponding to the expelling of a set dose, the first state being characterized by the piston being elastically deformed by the piston drive corresponding to a build-up of pressure in the cartridge. In this state the control system controlling the expelling stroke of the piston rod in accordance with a set dose of drug to be expelled will register that the piston rod has reached its calculated end-of-stroke position which in conventional systems would indicate that the set dose of drug had been successfully expelled from the cartridge through an attached flow conduit such as a subcutaneous needle. However, as shown in FIG. 3, at this point in time the elastically deformable piston is still deformed indicating that the pressure in the cartridge is still elevated and that actual expelling of the set dose of drug has not yet taken place. Under normal conditions with unobstructed flow through e.g. an attached needle the remaining drug will be expelled during a few seconds driven by the elastically deformable piston regaining its form, this corresponding to the state shown in FIG. 2. However, if the outlet in the end-of-stroke state as shown in FIG. 3 is occluded the piston will stay deformed (at least for a prolonged period of time), this indicating that the outlet is at least partially occluded. Occlusion may take place at the end of delivery of a larger dose, e.g. the remaining 3 IU of a 40 IU dose of insulin may not be expelled, however, this situation is not very likely to take place just as the importance of receiving 37 IU instead of 40 IU insulin may not be vital to the user. However, occlusion may also take place at the beginning of delivery of a smaller dose, e.g. the entire 3 IU of a 3 IU dose of insulin may not be expelled, this situation being more likely to happen (e.g. due to a defective or initially blocked needle) just as the importance of not receiving 3 IU out of 3 IU of insulin is by far more vital to the user.

Correspondingly, the controller 30 is configured to detect an end-of-stroke (or end-of-dose) state when the piston drive member has just been moved to the end-of-stroke position corresponding to the fully expelling of a set dose, the end-of-stroke state being characterized by the piston being elastically deformed by the piston drive member corresponding to a build-up of pressure in the cartridge. After a given amount of time (e.g. 3 seconds) an end-of-dose sensor value is determined based on light reflected from the proximal piston portion which is then compared with a reference value. Based on the difference between the end-of-dose sensor value and the reference value the controller determines whether the system is in (i) a non-occlusion state in which the elastically deformed piston has relaxed to such a degree that it is indicative of a reduction in the pressure build-up in the cartridge due to drug being expelled from the cartridge via a connected flow conduit, e.g. a needle, by energy stored in the elastically deformed piston, or (ii) an occlusion state in which the elastically deformed piston has failed to relax to such a degree that is can be determined as being indicative of a connected flow conduit being occluded. If an occlusion state is detected the controller may indicate this to the user by appropriate signal means like an acoustic, visual and/or vibrational alarm.

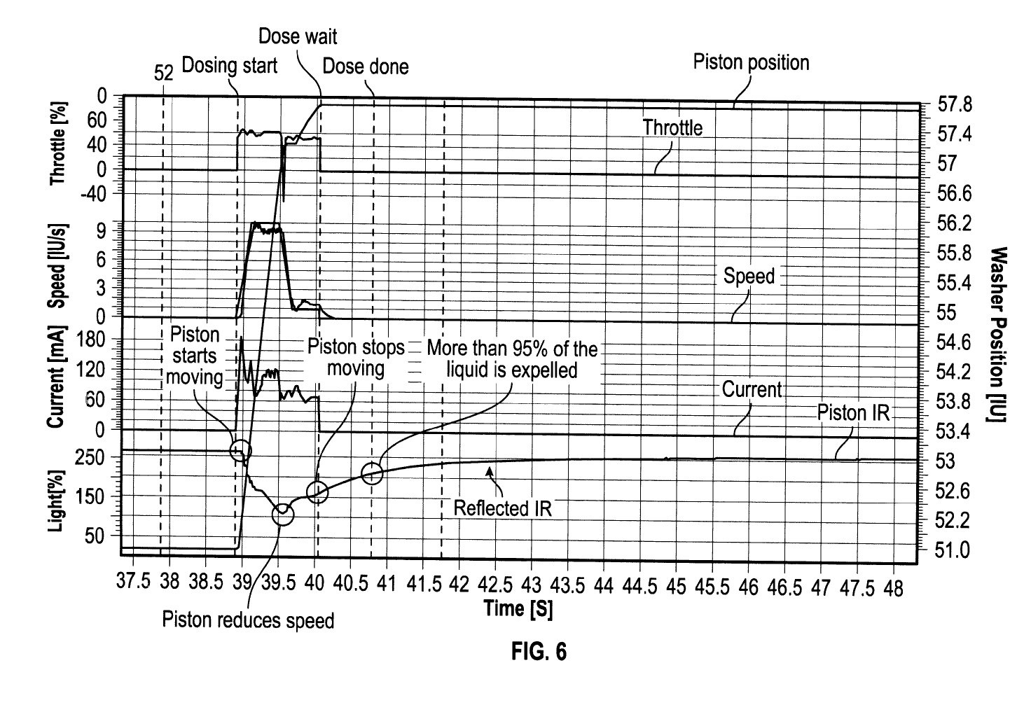

FIGS. 4-6 show experimental data for a set-up similar to the system shown in FIGS. 2 and 3. More specifically, the “piston rod” position corresponding to the washer distal surface 15 (see FIG. 1) is measured in IU corresponding to a standard 3 ml 100 IU/ml Novo Nordisk Penfill® insulin cartridge with zero corresponding to a loading position in which the piston is positioned in its proximal-most position. The figure shows “throttle”, “speed” and “current” for the motor drive as well as “piston IR” for measured light.

FIG. 4 shows infusion of a 1 IU dose injected in air. In the starting position at “Dosing start” the piston rod is positioned as shown in FIG. 2 allowing 2.2% of light to be detected. Piston proximity light level then reaches a minimum (0.5%) when the whole 1 IU is delivered at “state dose wait” (corresponding to the above-mentioned end-of-dose state) and then slowly raises, indicating a relaxation of the system. After 6 seconds at “state dose done” the light level is 1.6% which is above halfway back to the level before the injection (2.2%) and the injection is considered successful.

In the FIG. 4 example the following calculations are used to detect a non-occlusion state. A first measured reference value “ref1” is determined at the “Dosing start” point of time just prior to the begin of out-dosing, and a second measured reference value “ref2” is determined at the “state dose wait” point of time when the piston rod has just stopped moving. From FIG. 4 the values can be determined to be approximately ref1=2.2 and ref2=0.5. After 6 seconds an end-of-dose sensor value “L” is determined which from FIG. 4 can be determined to be approximately L=1.6.

In the shown example a blocking condition is determined if L-ref2>k (ref1-ref2), in which k is a constant indicating the expected level of reproduction of the initial reflexion level. In the shown example k=50% although ideally k=100%. The following conditions must also be met: L>ref2 and ref1>ref2. Otherwise an error condition is detected. In the shown embodiment 1.6−0.5=1.1>0.5 (2.2−0.5)=0.85 indicating the expected finding of a non-occlusion state. All sensor values mentioned refers to measurements by the same sensor.

The determination of reference values is used to compensate for variations in e.g. sensors, materials and mounting. The determination of further reference values could be used to compensate e.g. non-linear output from a sensor system.

FIG. 5 shows infusion of a 1 IU dose with a blocked needle attached. The light level decreases during the injection as before in FIG. 5 but does not rise again. After 6 seconds the light level is below 50% of the pre-dose level and a blocked needle condition is determined.

The algorithm used in the experiment according to FIG. 5 requires that the user waits the recommended 6 full seconds until the needle is withdrawn but it may be possible to shorten this period. Comparing FIGS. 4 and 5 indicates that it may be possible to detect a blocked needle during the first second, however, this have to be verified by more controlled experiments with varying back-pressures, needle sizes, piston variants, etc. The physical construction of the “sensor head” could also be tweaked to improve the sensitivity for piston deformation.

Correspondingly, FIG. 6 shows an example in accordance with an embodiment of the invention in which reflected light is measured after the “dose wait” state has been reached for a 6 IU dose. When the reflected IR light has reached a given level a “dose done” state is determined. In the shown embodiment 95% of the 6 IU have been expelled approximately 2 seconds after the end-of-dose state.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A method of detecting an end-of-dose condition in a pressurized system, comprising the steps of:
providing a system comprising:
a fluid-filled variable volume reservoir comprising an elastically deformable portion and an outlet, the elastically deformable portion having an initial configuration,
structure for pressurizing the interior of the reservoir to thereby expel fluid through the outlet, and
structure for measuring a property of the elastically deformable portion which varies with the internal pressure in the reservoir,
actuating the pressurizing structure to pressurize the reservoir to expel a desired amount of fluid by applying a force, thereby deforming the elastically deformable portion,
measuring, after the actuating step, a property related to the elastically deformable portion as it regains its initial configuration,
comparing a value for the measured property with one or more threshold values, and
providing a user with an indication:
when a given threshold value has been reached, and/or
when a given threshold value has not been reached within a given period of time.

2. A method as in claim 1, wherein:
an indication is provided that expelling of the desired amount of fluid has ended when the given threshold value has been reached, and/or
an indication is provided that an occlusion state applies to the reservoir outlet when a given threshold value has not been reached within a given period of time.

3. A method as in claim 1, wherein the one or more threshold values are determined based on a reference value being one of:
a value determined prior to pressurizing the reservoir,
a value determined at the end of the action pressurizing the reservoir.

4. A method as in claim 1, wherein the provided system further comprises:
a reflecting surface adapted to reflect light,
a light source directing light against the reflecting surface, and
a light sensor adapted to measure light from the light source reflected from the reflecting surface, a measured value varying with the deformation of the elastically deformable portion,
wherein the measured property is light reflected from the reflecting surface.

5. A method as in claim 4, wherein the reservoir of the provided system comprises:
a main portion having a general cylindrical configuration, and
an axially displaceable piston arranged in the main portion, the piston comprising:
at least a part of the elastically deformable portion, and
the reflecting surface.

6. A method as in claim 1, wherein the measured property is one of:
pressure in the reservoir,
tension in a part of the pressurizing structure.

7. A drug delivery device comprising:
a drug-filled cartridge in a loaded position or a compartment adapted to receive a drug-filled cartridge in a loaded position, the cartridge comprising a body portion, an axially displaceable elastically deformable piston with a proximal portion comprising a surface adapted to reflect light, and a distal outlet portion adapted to be arranged in fluid communication with a flow conduit,
an expelling assembly comprising an axially displaceable piston drive member adapted to engage the proximal piston portion of a loaded cartridge, the piston drive member being moveable in a distal direction to thereby expel drug from a loaded cartridge, the piston thereby being elastically deformed by the piston drive member from its initial configuration,
a light source providing a light directed at least in part against the proximal piston portion of a loaded cartridge,
a light sensor adapted to measure light from the light source reflected from the proximal piston portion, the light sensor being arranged to provide a measured value varying with the deformation of the piston,
a controller coupled to the light sensor,
wherein the controller, with a cartridge in a loaded position, is configured to:
detect an end-of-stroke state when the piston drive member has just been moved to a position corresponding to expelling of a set dose, the end-of-stroke state being characterized by the piston being elastically deformed by the piston drive member corresponding to a build-up of pressure in the cartridge,
after detection of the end-of-stroke state measure light from the light source reflected from the proximal piston portion as the piston regains its initial configuration,
compare a measured light value with one or more threshold values, and
provide, when a given threshold value has been reached, a user with an indication thereof, and/or
provide, when a given threshold value has not been reached within a given period of time, a user with an indication thereof.

8. A drug delivery device as in claim 7, wherein:
an indication is provided that expelling of the desired amount of fluid has ended when the given threshold value has been reached, and/or
an indication is provided that an occlusion state applies to the reservoir outlet when a given threshold value has not been reached within a given period of time

9. A drug delivery device as in claim 7, wherein the threshold is determined based on a reference value being one of:
a value determined prior to pressurizing the reservoir,
a value determined at the end of the action pressurizing the reservoir.

10. A drug delivery device as in claim 7, wherein the light source and the light sensor provide at least in part a proximity sensor system adapted to provide an output indicative of the distance between the proximal piston portion and the piston drive member.

11. A drug delivery device as in claim 7, wherein the expelling assembly is electronically controlled, the controller being coupled to the expelling assembly and adapted to control the expelling assembly in order to expel a set dose.

12. A drug delivery device as in claim 7, wherein the light source and/or the light sensor is/are coupled to and moves with the piston drive member.
